(19) United States
(12) Patent Application Publication
(10) Pub. No.: US 2023/0399601 A1
(43) Pub. Date: Dec. 14, 2023

(54) MICROFLUIDIC DEVICES AND METHODS FOR DENUDATION OF CELLS

(71) Applicant: Overture Life, Inc., New York, NY (US)

(72) Inventors: Lionel MATTHYS, Madrid (ES); Joana Alexandra SALES FIDALGO, Madrid (ES); Pablo CARASA RUIZ, Madrid (ES); Javier GUERRERO SANCHEZ, Madrid (ES)

(73) Assignee: Overture Life, Inc., New York, NY (US)

(21) Appl. No.: 18/032,072

(22) PCT Filed: Oct. 15, 2021
(86) PCT No.: PCT/US2021/055172

§ 371 Date: Apr. 14, 2023

Related U.S. Application Data
(60) Provisional application No. 63/092,750, filed on Oct. 16, 2020.

(51) Int. Cl. C12M 1/42; C12M 3/06; C12M 3/00

(52) U.S. Cl. CPC C12M 35/04; C12M 23/16; C12M 21/06

(57) ABSTRACT

Disclosed herein are microfluidic devices and methods for denudation of biological materials, such as oocytes and embryos. For example, the devices and methods achieve separation of oocytes from surrounding cumulus cells in a fully automated manner and without manual intervention. The devices can facilitate continuous uni-directional fluid flow, as well as a bi-directional fluid flow mechanism that closely mimics manual methods.

### CROSS-REFERENCE TO RELATED APPLICATIONS

This application claims the benefit of U.S. Provisional Patent Application No. 63/092,750, filed Oct. 16, 2020, which is incorporated by reference herein in its entirety.

### BACKGROUND

The assisted reproduction field makes use of various assisted reproduction technologies (ART) for the purpose of treating infertility in male and female patients. One technology involves microinjection of a single spermatozoa directly into a female gamete (egg/oocyte) to achieve fertilization in a petri dish. This technology requires prior processing of the female gamete prior to the microinjection procedure. As not all oocytes have the competence and maturity to be fertilized, oocytes must be processed and classified prior to fertilization to establish the maturity status of the oocytes. However, this classification requires the removal of the cumulus and corona cells surrounding the oocyte. This process is called oocyte denudation. Furthermore, denudation is clinically relevant prior to egg freezing for the purposes of fertility preservation.

### INCORPORATION BY REFERENCE

Each patent, publication, and non-patent literature cited in the application is hereby incorporated by reference in its entirety as if each was incorporated by reference individually.

### BRIEF DESCRIPTION OF THE FIGURES

FIG. 1 shows a perspective view of a device described herein.

FIG. 2 shows a top view of a device described herein.

FIG. 3 shows a bottom view of a device described herein.

FIG. 4 shows a schematic of a device described herein, including an inlet reservoir, an outlet or waste reservoir, and the pumps for introducing the fluids in the channel.

FIG. 5 shows a sectional view of the device described herein, showing the well, the outlet channel section, the inlet channel section, and the denudation cavity.

FIG. 6 shows a detailed sectional view of the device of FIG. 5 of the lower base of the well and the denudation cavity.

FIG. 7 shows a perspective view of the lower base of the well and the denudation cavity of a device described herein.

FIG. 8 shows another perspective view of the lower base of the well and the denudation cavity of a device described herein.

FIG. 9 shows a lower view of the base of denudation cavity and the base of the well, showing a preferable shape of said cavity.

FIG. 10 shows an upper view of the well with a conical funnel structure.

FIG. 11 shows a lower view of a device described herein.

FIG. 12 shows an upper perspective view of the device of FIG. 11.

FIG. 13 shows an expanded view of the device of FIGS. 11 and 12.

FIG. 14 shows a device described herein.

FIG. 15 shows results of a denudation procedure using a device of FIG. 14.

FIG. 16 shows results of a denudation procedure using a device of FIG. 14.

FIG. 17 shows a schematic sectional view of an alternative well of a device described herein.

FIG. 18 shows a schematic sectional view of a further alternative well of a device described herein.

FIG. 19 shows a schematic sectional view of the device with the well of FIG. 18.

FIG. 20 shows a schematic sectional view of an alternative well of a device described herein with only one outlet channel.

FIG. 21 shows a schematic sectional view of another alternative well of a device described herein with only one outlet channel.

### DETAILED DESCRIPTION

Disclosed herein are microfluidic chips, biochips, devices, and methods for denudation of cells, such as oocytes, zygotes, or embryos. For example, the microfluidic chips, biochips, devices, and methods can be used for removing cumulus cells (granulosa and corona cells) surrounding oocytes or zygotes. This process, termed “denudation,” is performed to obtain a denuded oocyte for the use in a clinical setting. Such method uses a microfluidic device setup, with an open well with a protrusion/cavity fluidically connected to a channel that extends through the lower part of the well and the protrusion/cavity. Devices and methods described herein are used for stripping surrounding cumulus cell mass to achieve high denudation efficiency and obtain a naked oocyte or zygote.

Devices and methods described herein avoid the use of manual pipetting, as well as exposure of the oocyte to enclosed channels that can, in the case of device failure, trap the oocyte. These devices and methods obtain a naked oocyte using a gentle protocol that results in less damage and improved development potential of the oocyte, as well as avoiding contamination of cumulus cells in the culture media for purposes of non-invasive preimplantation genetic diagnosis (NI-PGT), or proteomics or metabolomics analysis of embryo culture media.

Devices and methods described herein can be used to remove cumulus cells surrounding an oocyte or zygote. Oocytes containing cumulus cells is known as Cumulus Oocyte Complexes (COC). Devices described herein can be used for stripping, rubbing, or shearing surrounding cumulus cell mass to achieve high denudation efficiency and obtain a naked oocyte or zygote.

The devices and methods described herein can be applicable to the field of assisted reproduction for the treatment of infertility or for fertility preservation, specifically to devices and methods for automatically processing and separating Cumulus Oocyte Complexes (COC) to obtain naked oocytes for downstream use in the assisted reproduction applications.

### Oocyte Denudation

The assisted reproduction field makes use of various assisted reproduction technologies (ART) for the purpose of treating infertility in male and female patients. One technology, intracytoplasmic sperm injection (ICSI), involves microinjection of a single spermatozoa directly into a female gamete (egg/oocyte) to achieve fertilization in a petri dish. This technology requires prior processing of the female gamete prior to the microinjection procedure. As not all oocytes have the competence and maturity to be fertilized, oocytes must be processed and classified prior to fertilization to establish the maturity status of the oocytes. However, this classification requires the removal of the cumulus cells surrounding the oocyte. This process is called oocyte denudation.

After retrieval from the ovary of a female patient, e.g., by aspiration from the ovarian follicles using a needle, the female gametes (egg/oocyte) contain a surrounding mass of cells known as cumulus cells. Cumulus cells can be removed with a combination of enzymatic treatment and mechanical pipetting in a petri dish. Complete cumulus cell removal is required to avoid biological material contamination caused by cumulus cells for the purpose of testing embryo culture media via non-invasive pre-implantation genetic diagnosis (NI-PGT), proteomics, or metabolomics to select the most viable embryos.

For the last two decades, this method of cumulus cell removal has been used successfully in human in vitro fertilization (IVF) prior to freezing of patient oocytes (fertility preservation) and for the IC SI technique. However, this manual procedure often results in variations from operator to operator. Thus, automation and standardization of denudation methods can improve the consistency and reliability of these procedures.

For example, microfluidics can be used for mechanical removal of cumulus cells without enzyme exposure. Microfluidic devices can process one or more cells at a time and allows automatic controlling and switching of multiple fluid flows. For example, a device can include a microchannel configured to maintain continuous fluid flow as well as repeating constriction-expansion units and surface features to ensure complete oocyte denudation. However, microfluidic devices containing closed fluid microchannels can trap valuable cells in cases of blockage or malfunction without a manual retrieval mechanism.

In some assisted reproduction technology procedures, COCs as well as follicular fluid are retrieved from patient ovaries and placed in a petri dish. COCs are selected with a micropipette and placed in a separate dish for washing with bicarbonate buffered media. Subsequently, COCs are placed in micro-drops in a separate petri dish with a sperm sample for overnight insemination (e.g., IVF) in fertilization media. After fertilization, fertilized oocytes (now zygotes) are manually washed to remove any remaining cumulus cells and placed in drops of embryo culture media for 5-6 days where the zygotes are cultured under special temperature and CO₂/O₂ gas conditions. The above-mentioned washes are carried out with micropipettes and not with the aid of synthetic hyaluronidase enzyme due to the natural release of this enzyme by spermatozoa to break through the cumulus layer of cells to reach the oocyte.

Described herein are improved systems, devices, and methods of cumulus cells removal pre-insemination or at an optimal time post-insemination. These systems, devices, and methods utilize microfluidics (e.g., by automation, miniaturization, and fine flow control) and allows the localization of the cells in an open chamber/well during the denudation process. These microfluidic devices allow both continuous uni-directional fluid flow, as well as a back and forth, bi-directional fluid flow mechanism that closely mimics manual methods to achieve a gentler and more efficient denudation outcome.

### Microfluidic Devices and Methods

Described herein are devices and methods for denuding oocytes from surrounding cumulus cells with broad applications in the field of assisted reproduction and infertility treatment. The devices enable the denudation of an oocyte or zygote mechanically in an open well within the microfluidic device. The device can be a biochip or a microfluidic chip.

In some embodiments, a device for receiving and for the manipulation of cells or cell masses including, for example, eggs, ova, oocytes, zygotes, and embryos, contains:
a substrate with at least one channel with an inlet and an outlet for carrying a fluid;
a well with a lower base and an open upper end, positioned between the inlet and the outlet of the channel, to divide the channel into an inlet section channel and an outlet section channel, and the lower base is fluidically connected to and on top of the channel; and
a denudation cavity that is positioned on top of the channel, and have a geometry with two side walls, a ceiling, an end wall, an open end opposite to the end wall, and an open base, wherein the open end that is opposite the ceiling is fluidically connected to the well and the open base is fluidically connected to the outlet section channel.

In some embodiments, the well has a conical shape, and contains a narrow lower base and an open upper end. The well is positioned on top of the microfluidic channel between the inlet and the outlet, so that the fluid is injected or removed through the channel below the well. The well also contains a cavity with two ends, one opened end fluidically connected to the well for infusing media and an opposite closed end, thereby providing said cavity with a geometrical shape/feature used to perform denudation of the biological sample, where the media can move towards the well or towards the microfluidic channel outlet or the cavity.

The term “fluidically connected” can refer to the free movement of a fluid between two elements. Thus, the fluid can move between the well and the channel, between the well and the cavity, and between the cavity and the channel. In some embodiments, the three elements (the well, the channel, and the cavity) are fluidically connected in series such that fluid can freely flow through each of the three elements at the same time.

In some embodiments, the fluid is cell media, buffered media, culture media, or embryo culture media. In some embodiments, the fluid comprises hyaluronidase.

In some embodiments, the well has a conical shape with an open upper end and a lower end, or base, with a perimeter body connecting said base with the upper end. Protruding from the base of the perimeter body of the well is the denudation cavity. The cavity can have two ends: one end fluidically connected to the well and the other end closed. The well and the protrusion can be positioned above, and fluidically connected through the base of the well to the channel or microchannel. The cavity can contain a ceiling enclosing the upper side of the cavity. Therefore, the geometry of the cavity can comprise of two side walls, a ceiling, an end wall, an open end opposite to the end wall, and an open base that is opposite the ceiling. In some embodiments, the open end can be fluidically connected to the well and the open base can be fluidically connected to the channel or microchannel.

The substrate of the device can contain a first reservoir and a second reservoir or waste reservoir. The first reservoir is fluidically connected to the inlet. The second reservoir or waste reservoir is fluidically connected to the outlet.

In some embodiments, the open well is funnel (conical) shaped and configured to provide access to load or unload biological material through the upper open end of the well to the lower portion or base of the well that is connected to the channel. The denudation cavity can protrude out from the perimeter of the well towards the outlet of the channel being therefore placed over the section of channel that goes towards the outlet, therefore the outlet channel section.

The lower portion or base of the well can be fluidically connected to the channel, and thus, can be directly connected to the inlet and outlet of the channel, which are each situated at opposing ends of the channel. In some embodiments, the channel can be located at the lower base of the well. In other embodiments, the channel is not located at the lower base of the well.

In some embodiments, the size of said channel does not allow biological material to travel through the channel, as the cross-sectional area is narrower than the size of the COC or oocytes/zygotes. That is, the cross-sectional area of the channel can be dimensioned to lessen the likelihood that the cell passes through the channel. For example, the cross-sectional area of the channel can be dimensioned to prevent the cell from passing through the channel. In some embodiments, the device is configured to generate bi-directional flow of the fluid (or solution) between the well and the channel.

This denudation cavity allows the COC, carried by the media, to be stripped of cumulus cells around the oocyte by forcing the introduction of the COC to the denudation cavity. Introduction of the COC to the cavity causes the COC to rub against the entrance and inside walls and ceiling of the cavity, thereby detaching the cumulus cells from the oocyte. The bi-directional movement of the COC inside and outside of the cavity, carried by the media, denudes the oocyte.

Further, the open end of the well allows the deposition of a COC into the well and the subsequent retrieval of an oocyte or zygote. In other embodiments, the well can be heated to maintain a desired temperature inside the well.

In some embodiments, the geometry of the denudation cavity is a closed-end tunnel shape. The cavity can be fluidically connected through the lower base to the section of the channel towards the outlet. In this closed end tunnel shape, the open end of the tunnel can be fluidically connected to the well and the lower base of the tunnel can be fluidically connected to the channel that extends under the cavity.

The denudation cavity can be configured to effect denudation of a cell, e.g., the denudation cavity can be dimensioned to have approximately the same dimensions as the cell such that the walls of the cavity can exert mechanical shear stress onto the surface of the cell as the cell enters and exits the denudation cavity, thereby denuding the cell. In some embodiments, the denudation cavity has a dimension that is approximately the diameter of the cell. In some embodiments, the denudation cavity has two dimensions, wherein each of the two dimensions is approximately the diameter of the cell. In some embodiments, the denudation cavity is configured to denude the cell by mechanical shear stress caused by the cell moving in and out of the denudation cavity. In some embodiments, the denudation cavity has a textured surface to promote cell denudation.

Devices described herein can be used to partially denude or completely denude a cell. For example, a cell can be considered “partially denuded” if fertilization of the cell is successful (i.e., fertilization assessment), but at least some cumulus or corona cells remain on the surface of the cell. Partially denuded cells can be used for IC SI or vitrification procedures. In some embodiments, the pronucleus or polar bodies of a cell can be visible in a partially denuded cell. Alternatively, a cell can be considered “completely denuded” if no cumulus or corona cells remain on the surface of the cell. Completely denuded cells can be required for NI-PGT, and proteomics and metabolomics analysis. The degree of denudation of a cell can be assessed by visual analysis, for example, using a computer vision algorithm.

In some embodiments, the denudation cavity described herein exerts a shear force (or shear stress) on a cell that is lesser than that of other denudation techniques, e.g., manual denudation. In some embodiments, the denudation cavity exerts a shear force that is lesser than 100 pascals (Pa), lesser than 90 Pa, lesser than 80 Pa, lesser than 70 Pa, lesser than 60 Pa, lesser than 50 Pa, lesser than 40 Pa, lesser than 30 Pa, lesser than 20 Pa, lesser than 10 Pa, or lesser than 5 Pa. For example, the denudation cavity can exert a shear force that is about 1 Pa, about 2 Pa, about 3 Pa, about 4 Pa, about 5 Pa, about 6 Pa, about 7 Pa, about 8 Pa, about 9 Pa, about 10 Pa, about 11 Pa, about 12 Pa, about 13 Pa, about 14 Pa, about 15 Pa, about 16 Pa, about 17 Pa, about 18 Pa, about 19 Pa, or about 20 Pa. Shear force can be approximated based on fluid characteristics (e.g., flow rate and viscosity) within a denudation device, and the dimensions and geometry of the denudation device.

The denudation cavity can be configured to fit an oocyte (having an average size of approximately 150 μm) such that the oocyte cannot travel through the channel. In contrast, cumulus cells (waste material) having a small diameter than the oocyte and the channel can travel along the channel to the second reservoir through the outlet after being removed from the oocyte or zygote. A typical oocyte having an average size of 150 μm diameter cannot travel past the denudation geometry along the channel towards the outlet.

As described, the device can further comprise an additional channel that is in fluid communication with the open well and in fluid communication with the fluid reservoirs where fluids or media are stored on the substrate of the device. In some embodiments, the additional channel can be located at the lower base of the well. In other embodiments, the additional channel is not located at the lower base of the well. For example, the well can be placed on top of the channel, between the inlet and outlet of the channel, so that the first connection of the well with the inlet side of the channel is opposed to the connection of the well with the outlet side. Both connections between the well and the channel can be narrower, e.g., less than the diameter of an oocyte/zygote. These dimensions prevent the cells from entering the channel, while permitting smaller cumulus corona cells to pass through the channel. That is, the cross-sectional area of the channel can be dimensioned to lessen the likelihood that the cell passes through the channel. In some embodiments, the device is configured to generate bi-directional flow of the fluid (or solution) between the well, the inlet channel, and the outlet channel. In some embodiments, the inlet channel is on a plane that is higher than is a plane of the outlet channel.

The inlet and outlet of the channel, each connected to a different reservoir, can provide the means to connect to an external pneumatic tubing for pressure pump connection. Fluid injection in the device can be performed using programmable syringe pumps or pressurized air pumps. In some embodiments, the device can be used with syringe pumps instead of the combination of pressure pumps and reservoir.

The sections of the channel between the inlet and the well or the outlet and the well can have different shaped paths, straight, curved, or serpentine, for example, a serpentine channel with a straight region close to the well.

The inlet and outlet of the channel can be respectively connected to a first reservoir and to a second or waste reservoir. The first reservoir is independent from the second or waste reservoir. The reservoirs can be configured to store larger volumes of fluids or media. For example, the first reservoir that is connected to the inlet can store reagents. The second or waste reservoir that is connected to the outlet can store waste solutions. The volume of contents in the reservoirs can be controlled by pressure pumps, which modulate the pressure in the reservoirs and thus, the flow rate of the contents entering or leaving the channel. For example, the reservoirs can be connected with positive pressure and negative pressure pumps with pneumatic tubing. For embodiments that use syringe pumps, the syringe can serve as a reservoir and a capillary can be connected between the syringe and the channel. Non-limiting examples of types of pumps include syringe pumps and peristaltic pumps, which can directly connect fluidic capillaries between the pumps and the channel inlet and outlet.

The device can comprise more than one channel, respective wells, and respective denudation cavity. That is, the device configuration can be multiplied either by using several devices or chips simultaneously, or by including several wells and respective channels inside one single device or chip having common inlet and outlet reservoirs. In some embodiments, the channels are positioned in such a way that the inlets and outlets of each channel are aligned. By this disposition, one reservoir can be used for all the inlets and the other reservoir can be used for all the outlets. In addition, a mirror disposition can be used on the device or chip, thereby doubling the number of wells and respective channels. In this disposition, only one reservoir that is positioned in the middle is used for all the inlets, e.g., 16 inlets, two reservoirs are used for the outlets: one reservoir for the outlets serving, e.g., 8 wells on one side, and another reservoir for the outlets serving, e.g., the other 8 wells on the opposite side.

In some embodiments, a method for denuding oocytes comprises the following steps:
Filling the inlet section channel between the inlet and the well of a device described herein with a solution, e.g., media. For example, a positive pressure is exerted on the inlet reservoir to push the solution through the inlet of the channel to fill the well and the inlet section of the channel that is between the inlet and the well.
Filling the outlet section channel between the well and the outlet of the device with the solution. For example, a negative pressure can be exerted to pull the solution from the well through the outlet of the channel to fill the section of the channel between the well and the outlet. Alternatively, the outlet section of the channel, between the outlet and the well, can be filled by infusing a solution from the outlet reservoir using positive pressure.
Introducing a COC in the well through the open upper end of the well.
Alternating application of a negative pressure and a positive pressure through the outlet of the channel to generate a bi-directional movement of the COC between the denudation cavity and the well, thereby forcing the COC to enter and exit the denudation cavity.
Executing several cycles of the negative/positive pressure application until the oocyte is denuded.
Retrieving the oocyte from the well through the upper end of the well.

The protocol comprises three main actions: priming, bi-directional media application, and media replenishment.
During priming, a fluid or media is infused from the inlet reservoir to fill the inlet section of the channel and the well. Thereafter, the fluid or media is either withdrawn from the outlet reservoir to fill the outlet section of the channel or introduced from the outlet reservoirs, through the outlet and across the outlet section of the channel, towards the well.
In the bi-directional media application, the application of alternate positive and negative pressures generates a bi-directional movement of fluid in the outlet channel, and consequently, a bi-directional movement of the COC sample through the well/channel, thereby creating mechanical stress on the COC sample. Cumulus cells are removed by the rubbing of the COC against the walls and ceiling of the denudation cavity through this bi-directional movement through the cavity. In some embodiments, the negative pressure is greater than is the positive pressure such that the cavity exerts sufficient shear stress when the COC sample is pulled towards the outlet section of the channel and inside the denudation cavity to move the cumulus cells gradually through the outlet channel towards the waste reservoir. A lower positive pressure can ensure that the COC sample is released from the cavity and released again inside the well.
Media replenishment can be necessary due to the negative (withdrawal) pressure being higher than the positive pressure that is applied during the bi-directional action. As the protocol advances, the level of media inside the well can decrease due to this negative pressure. Therefore, after several cycles of bi-directional action, fresh media can be replenished. During media replenishment, media can be infused from the inlet channel to compensate the volume loss and a smaller pressure can be applied on the outlet section of the channel, from the outlet, to allow the removal of cumulus cells released inside the well.

Regarding the pressure magnitudes, the applied positive pressure can be sufficient to release the sample from the denudation feature, and applied negative pressure can be sufficient to pull back the sample towards the denudation feature. In some embodiments, each of the positive and negative pressures can be modulated and optimized to the protocol as desired. For example, alternating positive and negative pressure can be applied for at least 5 minutes (min), at least 10 min, at least 15 min, at least 20 min, at least 25 min, at least 30 min, at least 35 min, at least 40 min, at least 45 min, at least 50 min, at least 55 min, at least min, or more. In some embodiments, alternating positive and negative pressure can be applied for about 15 min to about 30 min. In view of the above, the single well device described herein can receive a COC and remove debris from the COC via the downstream outlet section of the channel. During denudation, the COCs are localized in the well and suspended in media. This configuration can allow for ease of visualization, e.g., using a microscope. Therefore, the device can allow automatic denudation of COCs by removing shred-off debris to yield a naked and cumulus-free oocyte.

In some embodiments, a liquid sample, e.g., media sample containing a COC, can be introduced into the open well of the device before the denudation procedure. During the denudation procedure, positive and negative pressure can be applied through the channels of the device to actuate alternating bi-directional fluid motion, e.g., an alternating flow direction. The bi-directional fluid motion can cause the COCs to enter through the denudation cavity of the device. As the COC cumulus mass traverses through the cavity, the COCs can rub against the edge, the walls, and/or ceiling of the cavity. As a result, the surrounding cells of the oocyte can be weakened and detached from the central oocyte. This process can be repeated many-fold until the oocyte is sufficiently denuded.

In some embodiments, the device described herein can also be used with an enzyme that is specific to cumulus cells, e.g., hyaluronidase. The enzyme can be added to a sample, e.g., media with COCs, to loosen the cumulus cells before introducing the COCs into the device. While the COCs remain confined to a single location in the device and subject to denudation using an in-situ denudation feature, debris can be removed through the waste channels.

Features and objectives of the devices and methods described herein include:
1) Localizing a COC in a given position within a device to reduce risk of damage or loss of the oocyte.
2) Automatically denuding oocytes from surrounding cumulus cells using a denudation cavity structure and a microfluidic protocol with a bi-directional movement that exerts significantly less mechanical stress than manual methods.
3) Removing cumulus cells through a waste channel to a waste reservoir.
4) Minimizing human intervention with reduced processing time and improving standardization of the procedure.
5) Ability to multiplex denudation features on a chip to increase throughput.

### Biological Materials

Devices and methods described herein can be used for any biological material, for example, cells, such as COCs, oocytes, zygotes, or embryos. In some embodiments, the biological material is human cells, such as human ova, oocytes, zygotes, or embryos. Devices and methods described herein can be used for biological material from any animal species. Non-limiting examples of biological material include stem cells, tissue cells, primate cells, bovine cells, murine cells, leporine cells, equine cells, swine cells, porcine cells, canine cells, and feline cells.

### EXAMPLES

### Example 1. A Microfluidic Device Described Herein and Uses Thereof

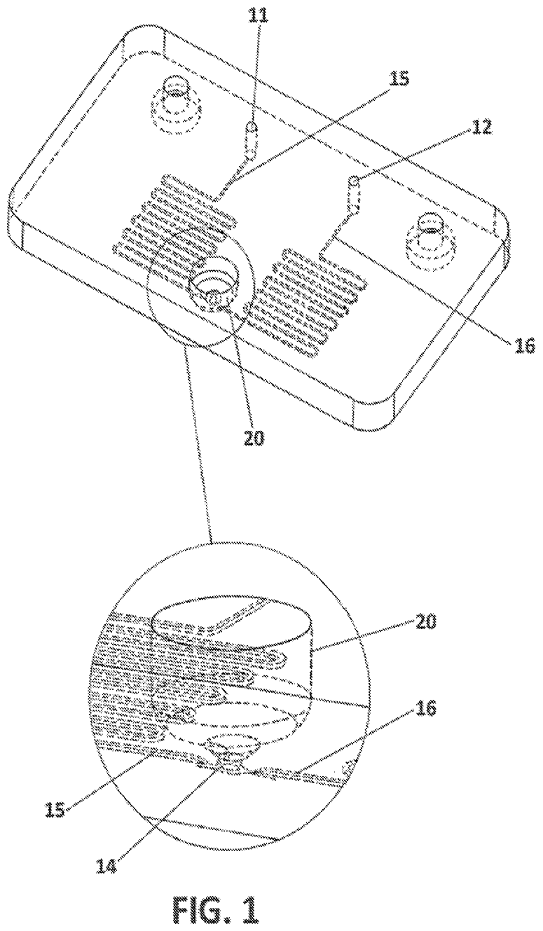

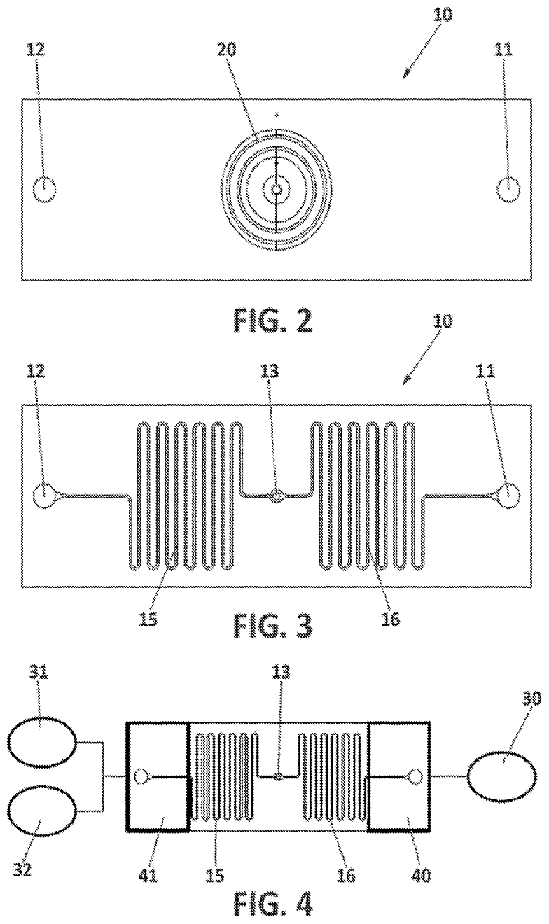

FIG. 1-4 refer to an example device described herein. The device comprises a substrate with at least one imprinted channel 15 and 16 with a respective outlet 12 and an inlet 11 for carrying a fluid or media through the channel. The device further comprises a well 20 that is between the outlet 12 and inlet 11 over the channel. The well 20 has a conical shape, including a narrow lower base 13 and an open upper end. The lower base 13 of the well is on top of the microfluidic channel 15 and 16, such that fluid can be injected or removed through the channel below the well. The channel has two sections, an inlet section channel 16 that is between the inlet 11 and the well 20, and an outlet section channel 15 that is between the well 20 and the outlet 12.

The well 20 is imprinted on one side, the upper side, of the substrate and the channel 15 and 16 on the opposite side, lower side, of the substrate. The outlet 12 or inlet 11 to the channel can be placed either on the upper or lower side of the substrate. The substrate comprises a layer on the lower side so that the section of the channel is closed, thereby allowing the liquid media or fluid to run along the channel.

FIG. 4 shows a schematic view of the device having two reservoirs, a first or inlet reservoir 40 connected to the inlet 11 and a second or outlet or waste reservoir 41 connected to the outlet 12. One positive pressure device 30 is connected to the inlet reservoir 40. At the same time, a second positive pressure device 31 and a negative pressure device 32 is connected to the waste reservoir 41. The positive pressure device 30 in the inlet 11 allows the introduction of liquid media through the inlet section channel 16. On the other hand, the negative pressure device 32 on the outlet side allows the extraction of media from the outlet 12 and the positive pressure device 31 on the outlet side allows the introduction of media from the outlet 12.

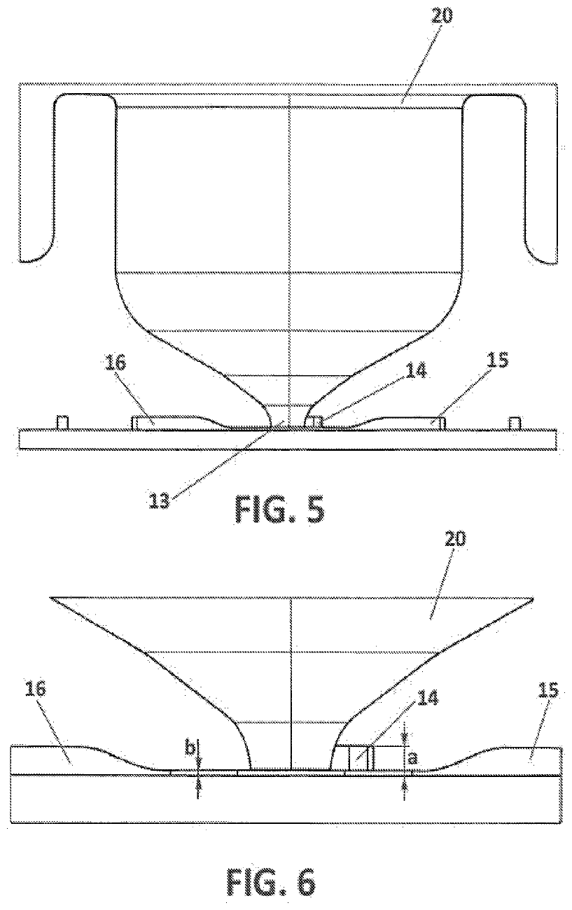

FIGS. 5 and 6 show a detail of a well 20, with an approximately conical shape, and a lower base 13 over the channel determined by the inlet channel section 16 and the outlet channel section 15. The lower base 13 is fluidically connected with the channel such that the well 20 and the channel are fluidically connected.

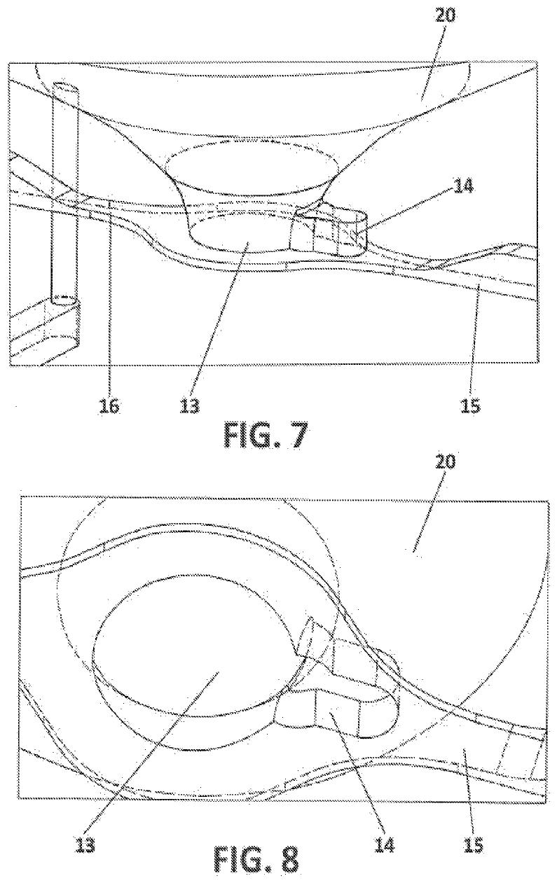

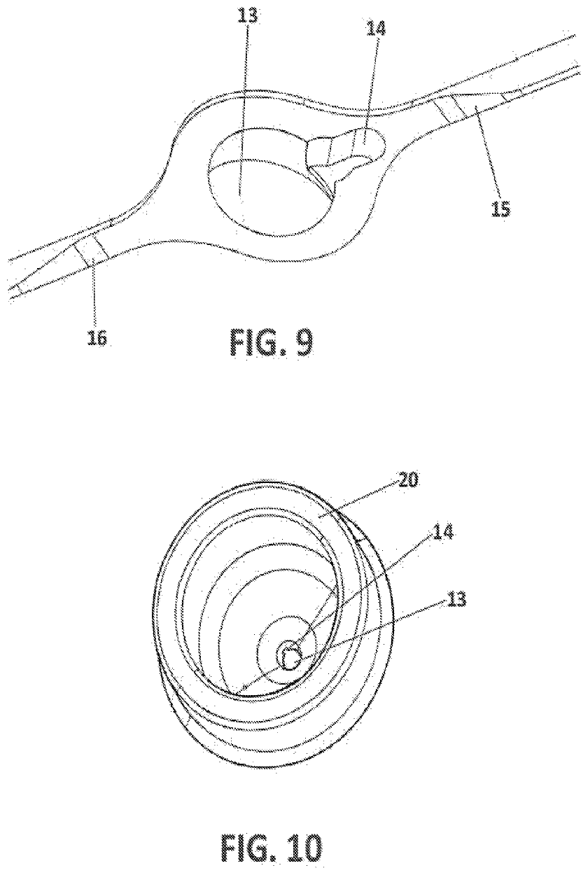

A denudation cavity 14 is positioned over the outlet channel section 15 and connected to the well 20. The geometry of the denudation cavity 14 (see FIG. 7-9) comprises two side walls, a ceiling, an end wall, an open end opposite to the end wall, and an open base that is opposite of the ceiling. The open end is fluidically connected to the well 20 and the open base is fluidically connected to the outlet section channel 15. The denudation cavity 14 has a shape that resembles a tunnel with one end closed.

The cross-sectional area of the cavity 14 is larger than the cross-sectional area of the channel 15 and 16, such that the height of the denudation cavity 14 is larger compared with the height of the channel 15 and 16. This difference allows the COC to move between the well 20 and the cavity 14, but not into the channel 15 and 16. The smaller sized channel 15 and 16, specifically, the outlet section channel 15, allows the cumulus cells or debris that are detached from the oocyte to travel along the outlet section channel 15 to the waste reservoir 41. FIG. 10 shows a top view of the well 20 with the denudation cavity 14 fluidically connected on one side of the well 20.

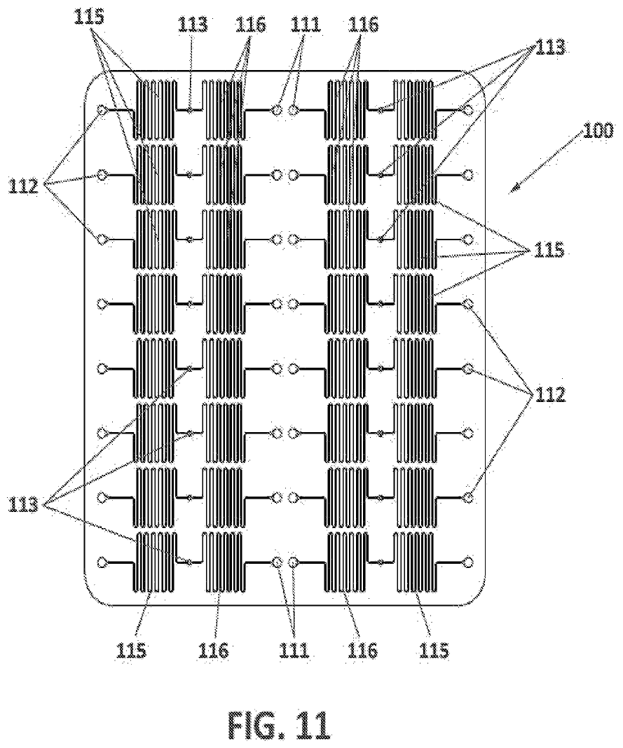

FIG. 11-13 show an alternative embodiment of a device 100. This embodiment comprises a device with more than one channel, each channel having a well and a denudation cavity, for example, sixteen channels, each having the well and the denudation cavity.

FIG. 11 shows a lower view of the device, where first eight channels are placed on one side of the substrate and another eight channels are placed in a mirror disposition opposite to the first eight channels. The inlets 111 of the channels are adjacent to one another and the outlets 112 are placed on the opposite sides. The wells 113 are positioned between the inlets 111 and the outlets 112, and between the inlet channel section 116 and the outlet channel section 115.

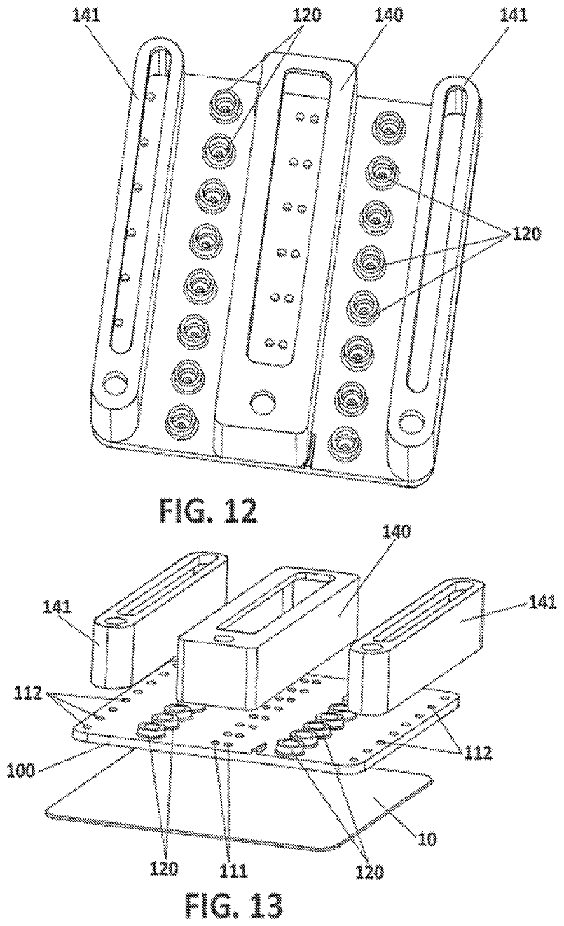

FIG. 12 shows a top view of the device 100 with an inlet reservoir 140 connected to the inlets 111 and two waste reservoirs 141 connected to the outlets 112. The upper side of the well 120 is also shown.

FIG. 13 shows an exploded view of the device 100 with an inlet reservoir 140 connected to the inlets 111 and two waste reservoirs 141 connected to the outlets 112, and a lower layer 10 for closing the lower side of the substrate.

Example dimensions of the elements of the device include:
Well bottom diameter: Between 0.4 and 0.6 mm, e.g., 0.5 mm
Well upper diameter: Between 0.4 and 0.6 mm, e.g., 5 mm
Well volume: at least 45 μl
Dimensions of the denudation cavity: Between 120 μm to 160 μm wide (W); between 120 μm to 160 μm height (H)×between 180 μm to 280 μm length (L), e.g., 150 μm (W)×145 μm (H)×250 (L)
Dimensions of the entry to the denudation cavity: Between 180 μm to 220 μm wide (W); between 120 μm to 180 μm height (H)×between 80 μm to 120 μm length (L), e.g., 200 μm (W)×100 μm (H)
Dimensions of the channel below the well and the denudation cavity: 400 μm (W)×between 20 μm to 60 μm, e.g., 30 μm (H)
Dimensions of the rest of the channel: 150-250 μm (W)×100-200 μm (L)×150-250 μm (H)

Due to the above elements and the disposition of the same in the device, the denudation of biological samples, such as COCs, can be achieved. The device allows the liquid media to flow between the outlet 12 and the well 20 (bi-directional movement) and between the well 20 and the cavity 14 or outlet section channel 15 (pull or back movement). The bi-directional movement of the COC between the well 20 and the denudation cavity 14 forces the COC to rub against the edges and walls of the denudation cavity 14, thereby mechanically agitating and removing the cumulus cells around the oocyte.

Continuous cycles of negative and positive pressure exerted in the outlet section channel 15, through the outlet 12, results in bi-directional fluid actions on the sample in combination with the geometry of the device. This method does not preclude the addition of enzymes, e.g., synthetic hyaluronidase, to the protocol, which can increase the denudation efficiency for complete denudation of COCs.

Example steps of the method include:
Filling the inlet section channel between the inlet and the well of a device as previously described;
Filling the outlet section channel between the well and the outlet of the device with a solution;
Introducing a COC in the well though the open upper end of the well;
Alternately applying a negative and a positive pressure, determining a cycle, through the outlet of the channel to generate a bi-directional movement of the COC between the denudation cavity and the well, thereby forcing the COC to enter and exit the denudation cavity;
Executing several cycles until the oocyte has been sufficiently denuded; and
Extracting the oocyte from the well through the upper end of the well.

### Example 2. A Denudation Protocol Using a Microfluidic Device Described Herein

In some embodiments, a denudation protocol comprises the following steps using the device shown in FIG. 1-10:
a) The fluid reservoirs are manually loaded with appropriate buffered media, such as culture media.
b) The channel or microchannel 15 and 16 of the device is primed with the media to achieve wetting of the channel. Priming is achieved by activating the positive pressure pump 30 to deliver fluid from the inlet reservoir 40 to the waste reservoir 41, passing through the well 20, or from both inlet 40 and outlet 41 reservoirs towards the well
c) The device containing the media in the channel 15 and 16, in the inlet section channel 16, and the outlet section channel 15, is equilibrated overnight in an incubator at 37° C. and established CO₂ and O₂ concentrations.
d) After a period of time, e.g., the next day, the device is removed from the incubator and a second priming is conducted to remove any accumulated bubbles in the channel 15 and 16 and the well 20.
e) After bubbles are removed, oocytes and spermatozoa samples are placed together into the well and incubated overnight in the established physiological conditions for fertilization to take place.
f) The next day, the bi-directional protocol is activated to begin the rinsing/washing of the oocyte to remove surrounding spermatozoa and cumulus cells.
g) Negative pressure is applied via the outlet channel 15 originating from the outlet external pumps 31 and 32 with the effect of pulling the zygote of interest towards the outlet channel 15 and into the denudation geometry 14 (FIGS. 4 and 5). Detached cumulus cells flow down the waste channel.
h) Subsequently, positive pressure is applied via the outlet channel 15 originating from the outlet pumps 31 and 32, which releases the zygote from the denudation geometry 14 and returns the zygote to the lower part of the well 13.
i) Steps g) and h) are repeated consecutively with alternating cycles of applied negative and positive pressure, thereby causing a bi-directional fluid motion for approximately 15-30 min to achieve denudation of the zygotes.
j) At the end of the denudation process, zygotes can be retrieved from the open well 20 by, e.g., using a manual stripper capillary.

Zygotes that may have been cultured on the chip post-fertilization, could additionally be submitted to a second denudation protocol in order to continue denudation with another series of bi-directional fluid flow cycles for appropriate period of time, in case that total denudation has not been achieved in a first round of denudation. This process could be performed to ensure that the zygote is completely stripped of surrounding cumulus cells that could be considered as contaminating in downstream analysis.

### Example 3. Automated Oocyte and Zygote Denudation Using a Microfluidic Device Described Herein

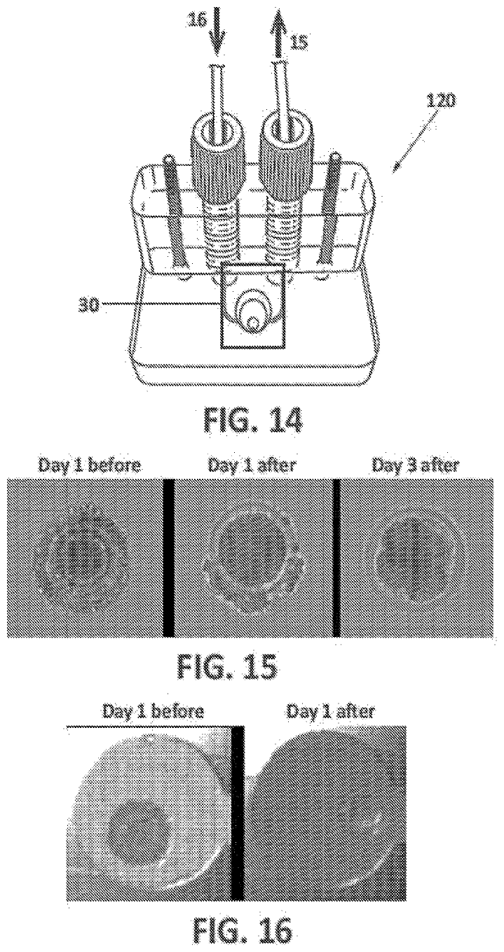

An automated cell denudation device, supervised by a computer vision algorithm, reduced shear stress while efficiently removed cumulus cells to allow vitrification and ICSI and for subsequent NI-PGT, proteomics, or metabolomics analysis. The automated denudation device is shown in FIG. 14. Experimental details regarding a comparative study using bovine and human cumulus-oocyte complexes in a microfluidic device 120 are shown in FIG. 15 and FIG. 16, respectively.

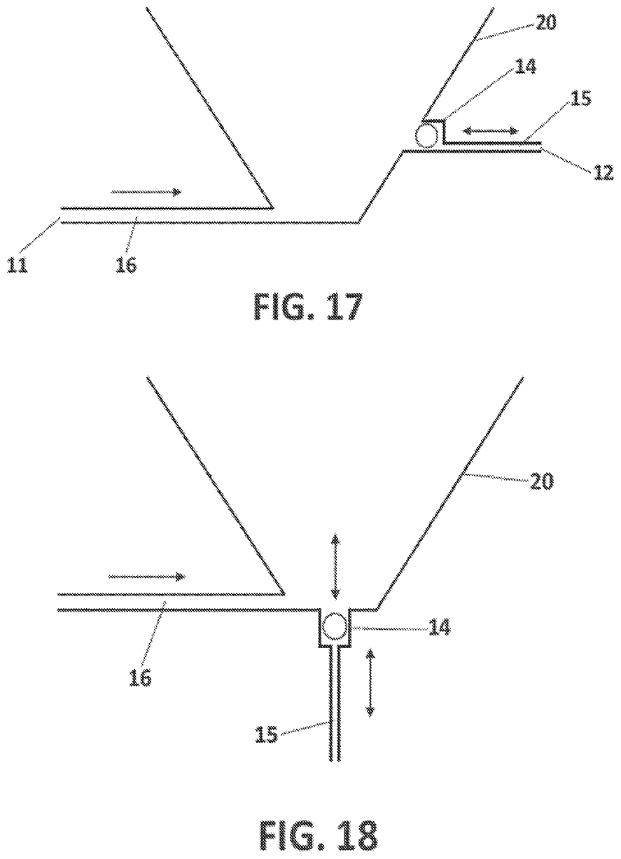

A microfluidic biochip 120 was developed and exerted a particular fluid motion while avoiding egg entrapment within the microfluidic channels of the biochip. In some embodiments, the fluid reservoirs are external reservoirs fluidically connected to the biochip. As shown in FIGS. 17 and 18, the microfluidic biochip 120 comprises an inlet channel 16, an outlet channel 15, and a well 20 with a denudation cavity 14. A microscope 30, not part of the device, was used for viewing the COCs in the well 20.

Firstly, cow cumulus oocyte complexes (COCs) were used due to size similarity to human COCs. Human COCs were later used. The COCs were either denuded 16-20 hours post insemination for 15 min or in a second denudation run on day 3 for NI-PGT, proteomics, or metabolomics analysis. Alternatively, COCs were denuded for 15 min pre-insemination. COCs were classified as “partially denuded” if fertilization assessment (ICSI or vitrification) was possible, or “completely denuded” if no cumulus cells remained (necessary for NI-PGT, proteomics, and metabolomics). Cow COCs controls were manually denuded (Stripper® pipette 145-μm ID) to compare shear stress between procedures. Experiments were repeated with the use of human COCs. A computer vision model was developed using human COCs to optically assess denudation efficiency. To obtain meaningful performance metrics, half of the entire dataset was defined as a test set of images, with a balanced ratio of denuded and non-denuded images. The model used was a PyTorch implementation of ResNet18 with ImageNet pretrained weights.

Results: 50 bovine COCs were microfluidically handled post insemination achieving complete ( 12/50) or partial ( 38/50) removal of the cumulus cells on day 1, while for day 3 double denudation group, 46 (92%) were completely denuded while the rest remained partially denuded. In comparison, 50/50 (100%) of manually denuded cow COC achieved complete denudation (post insemination group). In addition, 60% (N=10) cow COCs treated pre-insemination were partially denuded after 15 min of treatment while 100% were partially denuded after one hour of treatment. See FIG. 15.

Of 20 donated human COCs, 12 were denuded manually and 8 automatically. Those in the automatic group were all partially denuded enough to see Pronucleus (PN) and Polar Bodies (PBs). Based on flow rate, characteristics of the fluid, and the device dimensions and geometry, the shear stress of the example design described herein was approximated to be less than 4.4 Pa, about 10× lower than the one applied by the manual process (˜44 Pa). The deep learning algorithm was tested on 20 unseen human oocytes on day 1, with 10 true positives 9 true negatives, and 1 false negative (95% accuracy). See FIG. 16.

Complete denudation can help to avoid biological contamination for non-invasive PGT and proteomics or metabolomics analysis, while avoiding damage to the oocyte by excessive shear force. The automated system described herein efficiently denuded cow and human COCs with 10× less shear force than manual techniques and without human intervention. Using a computer vision algorithm, the device can recognize degrees of denudation to subsequently treat each oocyte individually.

### Example 4. A Microfluidic Device Described Herein and Uses Thereof

FIG. 17-19 refer to another example device described herein. The device comprises a substrate with at least one imprinted channel 15 and 16 with a respective outlet 12 and an inlet 11 for carrying a fluid or media through the channel. The device further comprises a well 20 that is between the outlet 12 and inlet 11 over the channel. The well 20 has a conical shape, including a narrower lower base 13 and a wider, open upper end. The section channel 16 is located at the lower base such that fluid can be injected or removed through channel 16 below well 20. The channel has two sections, an inlet section channel 16 that is between the inlet 11 and the well 20, and an outlet section channel 15 that is between the well 20 and the outlet 12.

As shown in FIG. 17-19, the device comprises:
an inlet microfluidic channel 16 for the introduction of a solution comprising an inlet 11 and an outlet;
an outlet microfluidic channel 15 comprising an inlet and an outlet 12;
a well 20 comprising a lower base, an open upper end, and a well inlet connected to the inlet channel 16, a well outlet connected to the outlet channel 15, wherein the well 20 is fluidically connected to both microfluidic channels 16 and 15 of the substrate; and
a denudation cavity 14, wherein the cavity is located at the perimeter of the well that is connected to the outlet channel 15 of the substrate, wherein the denudation cavity has a geometry with two side walls, a ceiling, an end wall, an open end opposite to the end wall, and an open side, e.g., opposite the ceiling, the open end being fluidically connected to the well 20 and the open side being fluidically connected to the outlet channel 15.

As shown in FIG. 17, inlet section channel 16 is on a plane that is lower than is the plane of outlet section channel 15. In some embodiments, inlet section channel 16 is on a plane that is higher than is the plane of outlet section channel 15. In some embodiments, inlet section channel 16 and outlet section channel 15 are each on the same plane. In some embodiments, inlet section channel 16 and outlet section channel 15 are not on the same plane.

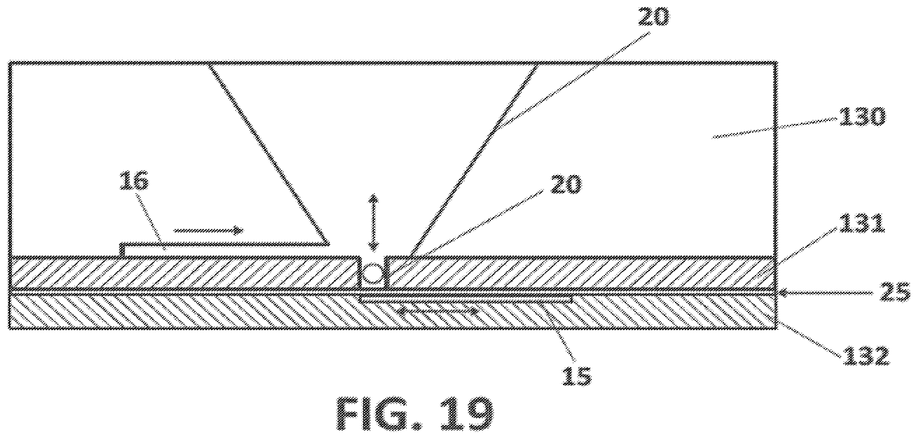

Further, in an embodiment shown in FIG. 17, the outlet channel 15 is positioned on a plane that is higher than is a plane of the inlet channel 16. Alternatively, in another embodiment in FIG. 18, the outlet channel 15 is positioned at the bottom of the well 20, perpendicular to the inlet channel 16. In some embodiments, as in FIG. 19, the well 20 and the inlet channel 16 are embedded in a first chip layer 130, while the denudation cavity 14 is embedded in a second chip layer 131 and the outlet channel 15 is embedded in a third chip layer 132. In some embodiments, a mesh or filter 25 is placed between the second layer 131 and third layer 132.

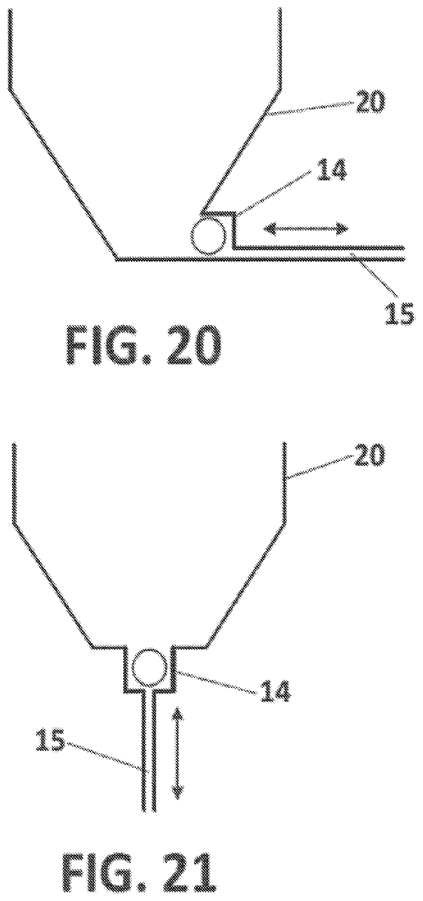

In alternative embodiments of the device, shown in FIGS. 20 and 21, the device comprises only outlet channel 15. Specifically, the device comprises:
a substrate comprising a single channel 15 with an inlet and an outlet configured to carry a fluid;
a well 20 comprising a lower base, an open upper end, a well outlet, wherein the well is fluidically connected to the outlet of the single channel 15; and
a denudation cavity 14, wherein the cavity is located between the well 20 and the channel 15 of the substrate, such that the channel is fluidically connected to the cavity 14 and the cavity 14 fluidically connected to the well 20. The geometry of the cavity comprises a closed body with an open side for fluidically connecting to the outlet channel 15 and an open side for fluidically connecting to the well 20.

FIG. 20 shows a device with only one outlet channel 15 fluidically connected to one side of the denudation cavity 14, that is also fluidically connected to one side of the base of the well 20. On an alternative construction, FIG. 21, the single outlet channel 15 is fluidically connected to the lower side of the denudation cavity 14, that is also fluidically connected to the lower end of the base of the well 20.

In these embodiments with a single outlet channel 15, the method for denuding oocytes, comprises the following steps:
priming the well 20 and the outlet channel 15 with a solution, e.g., media;
introducing a COC in the well 20 through the open upper end of the well 20;
alternating application of a negative pressure and a positive pressure through the outlet of the channel 15 to generate a bi-directional movement of the COC between the denudation cavity 14 and the well 20, thereby forcing the COC to enter and exit the denudation cavity 14;
executing several cycles of the negative/positive pressure application until the oocyte has been sufficiently denuded; and
extracting the oocyte from the well 20 through the upper end of the well 20.

Occasionally, between the cycles of pressure application, media replenishment of the well helps to prevent the well from being emptied, drying out, and potentially damaging the cell. The well can be replenished with media via the outlet of the channel 15.

### EMBODIMENTS

In some embodiments, a device comprises two channels (instead of one channel as described in Example 1): an inlet channel fluidically connecting the inlet and the well, and an outlet channel fluidically connecting the well and the outlet. Each channel comprises an inlet and an outlet. For the inlet channel, the inlet receives a fluid, and the outlet delivers the fluid to the well via the well inlet. For the outlet channel, the inlet receives fluid from the well via the well inlet, and the outlet expels fluid from the well. Different configurations of such a device are shown in FIG. 15-17.

Therefore, the device comprises:
an inlet microfluidic channel for the introduction of a solution comprising an inlet and
   an outlet;
an outlet microfluidic channel comprising an inlet and an outlet;
a well comprising a lower base, an open upper end, a well inlet connected to the inlet channel through its outlet, a well outlet connected to the inlet of the outlet channel, wherein the well is fluidically connected to both microfluidic channels of the substrate; and
a denudation cavity, wherein the cavity is on top of the outlet of the outlet channel of the substrate, with a geometry with two side walls, a ceiling, an end wall, an open end opposite to the end wall, and an open base, opposite the ceiling, the open end being fluidically connected to the well and the open base being fluidically connected to the outlet channel.

In some embodiments, the outlet channel is positioned on a plane that is higher than is a plane of the inlet channel (FIG. 15).

In some embodiments, the outlet channel is positioned at the bottom of the well, perpendicular to the inlet channel (FIGS. 16 and 17). In some embodiments, the outlet channel contains a mesh or filter (FIG. 17).

In some embodiments, the denudation cavity has a width that is shorter than the width of the base of the well and the denudation cavity has a height that is shorter than the width of the outlet channel.

In some embodiments, the denudation cavity has a height that is shorter than the height of the well, and has a height that is longer than the depth of the outlet channel.

In some embodiments, the inlet of the inlet channel is connected to a first reservoir and the inlet of the outlet channel is connected to a waste reservoir, said reservoirs placed on the substrate.

In some embodiments, the device comprises at least two fluid reservoirs. Each fluid reservoir comprises channels such that the channel inlets are adjacent to one another and share the same reservoir.

In some embodiments, the filling of the inlet channel is made by exerting a positive pressure to a solution introduced through the inlet channel to fill the section of the channel up to the height of the well.

In some embodiments, the filling of the outlet channel is made by exerting a negative pressure from the inlet of the outlet channel of the device to draw in the solution to fill the section of the outlet channel between the well and inlet of the outlet channel.

In some embodiments, the filling of the outlet channel is made by infusing a positive pressure to a solution introduced through the inlet of the outlet channel to fill the section of the outlet channel between the inlet of the outlet channel and the well.

In some embodiments, the positive pressure is lower than the negative pressure. In some embodiments, the method further comprises, after several cycles, introducing a new solution through the inlet channel to clean the well and refill the well with fresh media.

In some embodiments, the device comprises:
a substrate comprising a channel with an inlet and an outlet configured to carry a fluid;
a well comprising a lower base, an open upper end, a well inlet, a well outlet, wherein the well is between the inlet and the outlet of the channel of the substrate, wherein the lower base is fluidically connected to and placed on top of the channel of the substrate; and
a denudation cavity, wherein the cavity is on top of the channel of the substrate, with a geometry with two side walls, a ceiling, an end wall, an open end opposite to the end wall, and an open base, opposite the ceiling, the open end being fluidically connected to the well and the open base being fluidically connected to the outlet section channel.

In some embodiments, the denudation cavity has a smaller width than the base of the well and the denudation cavity has a smaller height than the width of the channel.

In some embodiments, the denudation cavity has a smaller height than the height of the well and a greater height than the depth of the channel.

In some embodiments, the inlet is connected to a first reservoir and the outlet is connected to a second or waste reservoir, said reservoirs placed on the substrate.

In some embodiments, the device comprises at least two wells, wherein each of the two wells comprise with respective channels such that the inlets are adjacent sharing the same reservoir and the outlets are adjacent, sharing the same waste reservoir.

In some embodiments, a method for denuding oocytes comprises:
a) priming the well and the channels with a solution;
b) introducing a cumulus oocyte complex (COC) in the well though the open upper end of the well;
c) alternating application of a negative pressure and a positive pressure through the outlet of the channel to generate a bi-directional movement of the COC between the denudation cavity and the well, thereby forcing the COC to enter and exit the denudation cavity;
d) executing several cycles of the negative/positive pressure application until the oocyte has been denuded;
e) washing the well or topping up the solution contents of well with additional solution from the inlet channel; and
f) extracting the oocyte from the well through the upper end of the well.

In some embodiments, the filling of the inlet section channel is made by exerting a positive pressure to a solution introduced through the inlet of a channel to fill the section of the channel up to the well.

In some embodiments, the filling of the outlet section channel is made by exerting a negative pressure from the outlet of the channel of the device to pull the solution to fill the section of the channel between the well and the outlet.

In some embodiments, the filling of the outlet section channel is made by infusing a positive pressure to a solution introduced through the outlet of the channel to fill said section of the channel between the well and the outlet.

In some embodiments, the positive pressure is lower than the negative pressure.

In some embodiments, the method further comprises, after several cycles, introducing a new solution through the inlet channel to clean the well and refill the well with fresh media.

In some embodiments, a device comprises:
a substrate comprising a single channel with an inlet and an outlet configured to carry a fluid;
a well comprising a lower base, an open upper end, a well outlet, wherein the well is fluidically connected to the outlet of the single channel of the substrate; and
a denudation cavity, wherein the cavity is situated at the entrance of the channel of the substrate, with a geometry with two side walls, a ceiling, an end wall, an open end opposite to the end wall, and an open base, opposite the ceiling, the open end being fluidically connected to the well and the base being fluidically connected to the outlet section channel. Example configurations of such a device are shown in FIG. 18.

In some embodiments, a method for denuding oocytes comprises:
a) priming the well and the channel with a solution;
b) introducing a cumulus oocyte complex (COC) in the well though the open upper end of the well;
c) alternating application of a negative pressure and a positive pressure through the outlet of the channel to generate a bi-directional movement of the COC between the denudation cavity and the well, thereby forcing the COC to enter and exit the denudation cavity;
d) executing several cycles of the negative/′positive pressure application until the oocyte has been denuded;
e) topping up the solution contents of well with additional solution from the outlet, of the channel; and
f) extracting the oocyte from the well through the upper end of the well

Embodiment 1. A device comprising:
(a) a well configured to receive a cell and a fluid at an open upper end, wherein the well is fluidically connected to an outlet channel, wherein the well comprises the open upper end, a lower base, a well outlet, a perimeter, and a denudation cavity;
(b) the outlet channel configured to expel the fluid from the well through the well outlet, wherein the outlet channel comprises an outlet channel inlet and an outlet channel outlet; and
(c) the denudation cavity configured to denude the cell, wherein the denudation cavity protrudes from the perimeter of the well towards the outlet channel.

Embodiment 2. The device of embodiment 1, wherein the denudation cavity has a dimension that is approximately a diameter of the cell.

Embodiment 3. The device of embodiment 1, wherein the denudation cavity has two dimensions, wherein each of the two dimensions is approximately a diameter of the cell.

Embodiment 4. The device of any one of embodiments 1-3, wherein the denudation cavity is configured to denude the cell by mechanical shear force caused by the cell moving in and out of the denudation cavity.

Embodiment 5. The device of embodiment 4, wherein the mechanical shear force is lesser than 50 Pa.

Embodiment 6. The device of embodiment 4, wherein the mechanical shear force is lesser than 10 Pa.

Embodiment 7. The device of any one of embodiments 1-6, wherein the device is configured to generate bi-directional flow of the fluid between the well and the outlet channel.

Embodiment 8. The device of any one of embodiments 1-7, wherein a cross-sectional area of the outlet channel is dimensioned to lessen a likelihood that the cell passes through the outlet channel.

Embodiment 9. The device of any one of embodiments 1-8, wherein the outlet channel is located at the lower base of the well.

Embodiment 10. The device of any one of embodiments 1-8, wherein the outlet channel is not located at the lower base of the well.

Embodiment 11. The device of any one of embodiments 1-10, wherein the outlet channel is a microfluidic channel.

Embodiment 12. The device of any one of embodiments 1-11, further comprising an inlet channel configured to introduce the fluid into the well through a well inlet, wherein the inlet channel comprises an inlet channel inlet and an inlet channel outlet.

Embodiment 13. The device of embodiment 12, wherein the device is configured to allow bi-directional flow of the fluid between the well, the outlet channel, and the inlet channel.

Embodiment 14. The device of embodiment 12 or 13, wherein a cross-sectional area of the inlet channel is dimensioned to lessen a likelihood that the cell passes through the inlet channel.

Embodiment 15. The device of any one of embodiments 12-14, wherein the inlet channel is located at the lower base of the well.

Embodiment 16. The device of any one of embodiments 12-14, wherein the inlet channel is not located at the lower base of the well.

Embodiment 17. The device of any one of embodiments 12-16, wherein the outlet channel is on a plane that is the same as a plane of the inlet channel.

Embodiment 18. The device of any one of embodiments 12-16, wherein the outlet channel is on a plane that is not the same as a plane of the inlet channel.

Embodiment 19. The device of any one of embodiments 12-18, wherein the inlet channel is a microfluidic channel.

Embodiment 20. The device of any one of embodiments 12-19, further comprising a storage reservoir configured to store the fluid, wherein the inlet channel is configured to introduce the fluid from the reservoir into the well.

Embodiment 21. The device of any one of embodiments 1-20, wherein the denudation cavity is configured to denude the cell from cumulus or corona cells.

Embodiment 22. The device of any one of embodiments 1-21, wherein the cell is a cumulus oocyte complex.

Embodiment 23. The device of any one of embodiments 1-22, wherein the cell is an oocyte comprising cumulus or corona cells on the surface of the oocyte.

Embodiment 24. The device of embodiment 23, wherein the outlet channel is configured to expel cumulus or corona cells from the well.

Embodiment 25. The device of any one of embodiments 1-24, further comprising a waste reservoir configured to store waste, wherein the outlet channel is configured to expel the waste from the well to the waste reservoir.

Embodiment 26. The device of embodiment 25, wherein the outlet channel is configured to expel cumulus or corona cells from the well into the waste reservoir.

Embodiment 27. The device of any one of embodiments 1-26, wherein the fluid is cell media.

Embodiment 28. The device of any one of embodiments 1-27, wherein the fluid is embryo culture media.

Embodiment 29. The device of any one of embodiments 1-28, wherein the fluid comprises hyaluronidase.

Embodiment 30. The device of any one of embodiments 1-29, wherein the well is conical shaped.

Embodiment 31. The device of any one of embodiments 1-30, wherein the device is a microfluidic chip.

Embodiment 32. A method for denuding a cell, the method comprising:
(a) filling a well of a device with a fluid, wherein the device comprises:
the well configured to receive the cell and the fluid at an open upper end, wherein the well is fluidically connected to an outlet channel, wherein the well comprises the open upper end, a lower base, a well outlet, and a denudation cavity;
the outlet channel configured to expel the fluid from the well through the well outlet, wherein the outlet channel comprises an outlet channel inlet and an outlet channel outlet; and
the denudation cavity configured to denude the cell, wherein the denudation cavity protrudes from the perimeter of the well towards the outlet channel;
(b) introducing into the upper end of the well the cell; and
(c) alternatively applying negative pressure and positive pressure within the device to generate a bi-directional flow of the fluid between the well and the outlet channel to transport the cell in and out of the denudation cavity, thereby denuding the cell.

Embodiment 33. The method of embodiment 32, wherein the alternative application of negative pressure and positive pressure within the device is applied until the cell is completely denuded as determined by visual analysis.

Embodiment 34. The method of embodiment 33, wherein the visual analysis is by a computer vision algorithm.

Embodiment 35. The method of any one of embodiments 32-34, wherein the alternative application of negative pressure and positive pressure within the device is applied for at least 15 minutes.

Embodiment 36. The method of any one of embodiments 32-34, wherein the alternative application of negative pressure and positive pressure within the device is applied for at least 30 minutes.

Embodiment 37. The method of any one of embodiments 32-34, wherein the alternative application of negative pressure and positive pressure within the device occurs for about 15 minutes to about 30 minutes.

Embodiment 38. The method of any one of embodiments 32-37, further comprising priming the well and the outlet channel with the fluid prior to (a).

Embodiment 39. The method of any one of embodiments 32-38, further comprising retrieving the denuded cell from the upper end of the well.

Embodiment 40. The method of any one of embodiments 32-39, wherein the denudation cavity has a dimension that is approximately a diameter of the cell.

Embodiment 41. The method of any one of embodiments 32-39, wherein the denudation cavity has two dimensions, wherein each of the two dimensions is approximately a diameter of the cell.

Embodiment 42. The method of any one of embodiments 32-41, wherein the denudation cavity is configured to denude the cell by mechanical shear force caused by the cell moving in and out of the denudation cavity.

Embodiment 43. The method of embodiment 42, wherein the mechanical shear force is lesser than 50 Pa.

Embodiment 44. The method of embodiment 42, wherein the mechanical shear force is lesser than 10 Pa.

Embodiment 45. The method of any one of embodiments 32-44, wherein the device is configured to generate bi-directional flow of the fluid between the well and the outlet channel.

Embodiment 46. The method of any one of embodiments 32-45, wherein a cross-sectional area of the outlet channel is dimensioned to lessen a likelihood that the cell passes through the outlet channel.

Embodiment 47. The method of any one of embodiments 32-46, wherein the outlet channel is located at the lower base of the well.

Embodiment 48. The method of any one of embodiments 32-46, wherein the outlet channel is not located at the lower base of the well.

Embodiment 49. The method of any one of embodiments 32-48, wherein the outlet channel is a microfluidic channel.

Embodiment 50. The method of any one of embodiments 32-49, wherein the device further comprises an inlet channel configured to introduce the fluid into the well through a well inlet, wherein the inlet channel comprises an inlet channel inlet and an inlet channel outlet.

Embodiment 51. The method of embodiment 50, wherein the device is configured to allow bi-directional flow of the fluid between the well, the outlet channel, and the inlet channel.

Embodiment 52. The method of embodiment 50 or 51, wherein a cross-sectional area of the inlet channel is dimensioned to lessen a likelihood that the cell passes through the inlet channel.

Embodiment 53. The method of any one of embodiments 50-52, wherein the inlet channel is located at the lower base of the well.

Embodiment 54. The method of any one of embodiments 50-52, wherein the inlet channel is not located at the lower base of the well.

Embodiment 55. The method of any one of embodiments 50-54, wherein the outlet channel is on a plane that is the same as a plane of the inlet channel.

Embodiment 56. The method of any one of embodiments 50-54, wherein the outlet channel is on a plane that is not the same as a plane of the inlet channel.

Embodiment 57. The method of any one of embodiments 50-56, wherein the inlet channel is a microfluidic channel.

Embodiment 58. The method of any one of embodiments 50-57, further comprising priming the well, the outlet channel, and the inlet channel with the fluid prior to (a).

Embodiment 59. The method of any one of embodiments 32-58, wherein the device further comprises a storage reservoir configured to store the fluid, wherein the inlet channel is configured to introduce the fluid from the reservoir into the well.

Embodiment 60. The method of any one of embodiments 32-59, wherein the denudation cavity is configured to denude the cell from cumulus or corona cells.

Embodiment 61. The method of any one of embodiments 32-60, wherein the cell is a cumulus oocyte complex.

Embodiment 62. The method of any one of embodiments 32-61, wherein the cell is an oocyte comprising cumulus or corona cells on the surface of the oocyte.

Embodiment 63. The method of any one of embodiments 32-62, wherein the outlet channel is configured to expel cumulus or corona cells from the well.

Embodiment 64. The method of any one of embodiments 32-63, wherein the device further comprises a waste reservoir configured to store waste, wherein the outlet channel is configured to expel the waste from the well to the waste reservoir.

Embodiment 65. The method of any one of embodiments 32-64, wherein the outlet channel is configured to expel cumulus or corona cells from the well into the waste reservoir.

Embodiment 66. The method of any one of embodiments 32-65, wherein the fluid is cell media.

Embodiment 67. The method of any one of embodiments 32-66, wherein the fluid is embryo culture media.

Embodiment 68. The method of any one of embodiments 32-67, wherein the fluid comprises hyaluronidase.

Embodiment 69. The method of any one of embodiments 32-68, wherein the well is conical shaped.

Embodiment 70. The method of any one of embodiments 32-69, wherein the device is a microfluidic chip.

What is claimed is:

1. A device comprising:
(a) a well configured to receive a cell and a fluid at an open upper end, wherein the well is fluidically connected to an outlet channel, wherein the well comprises the open upper end, a lower base, a well outlet, a perimeter, and a denudation cavity;
(b) the outlet channel configured to expel the fluid from the well through the well outlet, wherein the outlet channel comprises an outlet channel inlet and an outlet channel outlet; and
(c) the denudation cavity configured to denude the cell, wherein the denudation cavity protrudes from the perimeter of the well towards the outlet channel.

2. The device of claim 1, wherein the denudation cavity has a dimension that is approximately a diameter of the cell.

3. The device of claim 1, wherein the denudation cavity has two dimensions, wherein each of the two dimensions is approximately a diameter of the cell.

4. The device of claim 1, wherein the denudation cavity is configured to denude the cell by mechanical shear force caused by the cell moving in and out of the denudation cavity.

5. The device of claim 4, wherein the mechanical shear force is lesser than 50 Pa.

6. The device of claim 4, wherein the mechanical shear force is lesser than 10 Pa.

7. The device of claim 1, wherein the device is configured to generate bi-directional flow of the fluid between the well and the outlet channel.

8. The device of claim 1, wherein a cross-sectional area of the outlet channel is dimensioned to lessen a likelihood that the cell passes through the outlet channel.

9. The device of claim 1, wherein the outlet channel is located at the lower base of the well.

10. The device of claim 1, wherein the outlet channel is not located at the lower base of the well.

11. The device of claim 1, wherein the outlet channel is a microfluidic channel.

12. The device of claim 1, further comprising an inlet channel configured to introduce the fluid into the well through a well inlet, wherein the inlet channel comprises an inlet channel inlet and an inlet channel outlet.

13. The device of claim 12, wherein the device is configured to allow bi-directional flow of the fluid between the well, the outlet channel, and the inlet channel.

14. The device of claim 12, wherein a cross-sectional area of the inlet channel is dimensioned to lessen a likelihood that the cell passes through the inlet channel.

15. The device of claim 12, wherein the inlet channel is located at the lower base of the well.

16. The device of claim 12, wherein the inlet channel is not located at the lower base of the well.

17. The device of claim 12, wherein the outlet channel is on a plane that is the same as a plane of the inlet channel.

18. The device of claim 12, wherein the outlet channel is on a plane that is not the same as a plane of the inlet channel.

19. The device of claim 12, wherein the inlet channel is a microfluidic channel.

20. The device of claim 12, further comprising a storage reservoir configured to store the fluid, wherein the inlet channel is configured to introduce the fluid from the reservoir into the well.

21. The device of claim 1, wherein the denudation cavity is configured to denude the cell from cumulus or corona cells.

22. The device of claim 1, wherein the cell is a cumulus oocyte complex.

23. The device of claim 1, wherein the cell is an oocyte comprising cumulus or corona cells on the surface of the oocyte.

24. The device of claim 23, wherein the outlet channel is configured to expel cumulus or corona cells from the well.

25. The device of claim 1, further comprising a waste reservoir configured to store waste, wherein the outlet channel is configured to expel the waste from the well to the waste reservoir.

26. The device of claim 25, wherein the outlet channel is configured to expel cumulus or corona cells from the well into the waste reservoir.

27. The device of claim 1, wherein the fluid is cell media.

28. The device of claim 1, wherein the fluid is embryo culture media.

29. The device of claim 1, wherein the fluid comprises hyaluronidase.

30. The device of claim 1, wherein the well is conical shaped.

31. The device of claim 1, wherein the device is a microfluidic chip.

32. A method for denuding a cell, the method comprising:
(a) filling a well of a device with a fluid,
wherein the device comprises:
the well configured to receive the cell and the fluid at an open upper end, wherein the well is fluidically connected to an outlet channel, wherein the well comprises the open upper end, a lower base, a well outlet, and a denudation cavity;
the outlet channel configured to expel the fluid from the well through the well outlet, wherein the outlet channel comprises an outlet channel inlet and an outlet channel outlet; and
the denudation cavity configured to denude the cell, wherein the denudation cavity protrudes from the perimeter of the well towards the outlet channel;
(b) introducing into the upper end of the well the cell; and
(c) alternatively applying negative pressure and positive pressure within the device to generate a bi-directional flow of the fluid between the well and the outlet channel to transport the cell in and out of the denudation cavity, thereby denuding the cell.

33. The method of claim 32, wherein the alternative application of negative pressure and positive pressure within the device is applied until the cell is completely denuded as determined by visual analysis.

34. The method of claim 33, wherein the visual analysis is by a computer vision algorithm.

35. The method of claim 32, wherein the alternative application of negative pressure and positive pressure within the device is applied for at least 15 minutes.

36. The method of claim 32, wherein the alternative application of negative pressure and positive pressure within the device is applied for at least 30 minutes.

37. The method of claim 32, wherein the alternative application of negative pressure and positive pressure within the device occurs for about 15 minutes to about 30 minutes.

38. The method of claim 32, further comprising priming the well and the outlet channel with the fluid prior to a).

39. The method of claim 32, further comprising retrieving the denuded cell from the upper end of the well.

40. The method of claim 32, wherein the denudation cavity has a dimension that is approximately a diameter of the cell.

41. The method of claim 32, wherein the denudation cavity has two dimensions, wherein each of the two dimensions is approximately a diameter of the cell.

42. The method of claim 32, wherein the denudation cavity is configured to denude the cell by mechanical shear force caused by the cell moving in and out of the denudation cavity.

43. The method of claim 42, wherein the mechanical shear force is lesser than 50 Pa.

44. The method of claim 42, wherein the mechanical shear force is lesser than 10 Pa.

45. The method of claim 32, wherein the device is configured to generate bi-directional flow of the fluid between the well and the outlet channel.

46. The method of claim 32, wherein a cross-sectional area of the outlet channel is dimensioned to lessen a likelihood that the cell passes through the outlet channel.

47. The method of claim 32, wherein the outlet channel is located at the lower base of the well.

48. The method of claim 32, wherein the outlet channel is not located at the lower base of the well.

49. The method of claim 32, wherein the outlet channel is a microfluidic channel.

50. The method of claim 32, wherein the device further comprises an inlet channel configured to introduce the fluid into the well through a well inlet, wherein the inlet channel comprises an inlet channel inlet and an inlet channel outlet.

51. The method of claim 50, wherein the device is configured to allow bi-directional flow of the fluid between the well, the outlet channel, and the inlet channel.

52. The method of claim 50, wherein a cross-sectional area of the inlet channel is dimensioned to lessen a likelihood that the cell passes through the inlet channel.

53. The method of claim 50, wherein the inlet channel is located at the lower base of the well.

54. The method of claim 50, wherein the inlet channel is not located at the lower base of the well.

55. The method of claim 50, wherein the outlet channel is on a plane that is the same as a plane of the inlet channel.

56. The method of claim 50, wherein the outlet channel is on a plane that is not the same as a plane of the inlet channel.

57. The method of claim 50, wherein the inlet channel is a microfluidic channel.

58. The method of claim 50, further comprising priming the well, the outlet channel, and the inlet channel with the fluid prior to (a).

59. The method of claim 32, wherein the device further comprises a storage reservoir configured to store the fluid, wherein the inlet channel is configured to introduce the fluid from the reservoir into the well.

60. The method of claim 32, wherein the denudation cavity is configured to denude the cell from cumulus or corona cells.

61. The method of claim 32, wherein the cell is a cumulus oocyte complex.

62. The method of claim 32, wherein the cell is an oocyte comprising cumulus or corona cells on the surface of the oocyte.

63. The method of claim 32, wherein the outlet channel is configured to expel cumulus or corona cells from the well.

64. The method of claim 32, wherein the device further comprises a waste reservoir configured to store waste, wherein the outlet channel is configured to expel the waste from the well to the waste reservoir.

65. The method of claim 32, wherein the outlet channel is configured to expel cumulus or corona cells from the well into the waste reservoir.

66. The method of claim 32, wherein the fluid is cell media.

67. The method of claim 32, wherein the fluid is embryo culture media.

68. The method of claim 32, wherein the fluid comprises hyaluronidase.

69. The method of claim 32, wherein the well is conical shaped.

70. The method of claim 32, wherein the device is a microfluidic chip.
